(19) United States
(12) Patent Application Publication
(10) Pub. No.: US 2012/0140878 A1
(43) Pub. Date: Jun. 7, 2012

(54) PROCESS AND SYSTEM FOR GENERATION OF TOMOSYNTHESIS IMAGES WITH BLUR REDUCTION

(76) Inventor: Henri Souchay, Versailles (FR)

(21) Appl. No.: 13/307,379

(22) Filed: Nov. 30, 2011

(30) Foreign Application Priority Data
Dec. 1, 2010 (FR) 1059963

(51) Int. Cl. A61B 6/02

(52) U.S. Cl. 378/22

(57) ABSTRACT

A method for imaging a series of medical images by tomosynthesis with blur reduction with an imaging system comprising a radiation source configured to emit a total radiation dose, a detector, and a control unit configured to control the source is provided. The method comprises positioning, with the control unit, the source in at least two positions relative to the detector. The method also comprises emitting an individual radiation dose at least partially detected by the detector in each of the at least two positions. The method further comprises distributing, with the control unit, the total radiation dose among the individual radiation doses such that the strongest individual radiation doses are emitted by the source in positions corresponding to the beginning of an imaging session of the series of medical images.

### BACKGROUND OF THE INVENTION

1. Field of the Invention

Embodiments of the present invention relate generally to tomosynthesis radiography, and specifically to breast tomosynthesis. More particularly, embodiments of the present invention relate to methods for imaging a series of medical images by tomosynthesis with blur reduction by means of an imaging system comprising a radiation source, a detector and a control unit.

2. Description of the Prior Art

In breast tomosynthesis, several images of a breast held in position are acquired for different positions of an X-ray source of an imaging system relative to an X-ray detector of the imaging system held in place. The breast is usually positioned on a breast support in which the detector of the imaging system is placed. The breast is then compressed by a compression pad. Next, several images are acquired with the source shifting from one start position to a stop position, the breast, the support and the pad remaining in position. The source, when shifted from one position to another, describes rotation about a point located on the detector, generally the centre of its edge facing the patient.

A three-dimensional (3D) image of the breast is then reconstructed from the acquired images. The quality of the reconstruction depends on the angle of opening (angle between the two end positions of the source) and on the number of acquired images.

Conventionally, the total radiation dose received by the patient stays in the same order of magnitude as the radiation dose received by the patient during conventional bidimensional (2D) radiography. The total radiation dose received is distributed uniformly for all acquisition positions of the radiation source.

One disadvantage of this tomosynthesis method is that it does not enable an adequate resolution for detecting small anomalies such as microcalcifications. In fact, on the 3D image of the breast, the 3D zones corresponding to the microcalcifications suffer from a blurred patch function caused by the reconstruction algorithm.

A solution to this problem was proposed in the document FR 2 905 256 in which distribution of the individual doses of radiation is not uniform among the different positions of the radiation source. In the method described by this document, a strong dose is emitted by the source when it is in the position, hereafter perpendicular position, where the main direction of radiation is perpendicular to the surface of the detector. So, weighting between the information from the medical images acquired is done. A heavier weight is given to the image for which fine resolution can be obtained. For the other positions and especially those which lie away from the perpendicular position, the individual radiation dose is minimal. But this suffices to reconstruct a 3D image of the breast. In fact, the medical images corresponding to the positions which lie away from the perpendicular position give information on large objects, so a low dose is adequate, whereas medical images corresponding to the positions near the perpendicular position give information on details. If a strong individual radiation dose were not supplied, these details would have been smoothed out by the other medical images during 3D reconstruction of the breast.

However, the solution given by FR 2 905 256 fails to resolve the problem of blurring caused by movements made by the patient. In fact, movements made by the patient generate a blurred medical image which will degrade the quality of the 3D image reconstructed from the medical image.

Now, the introduced blur reduces the resolution of the 3D image and thus impedes identification of microcalcifications.

### BRIEF SUMMARY OF THE INVENTION

According to an embodiment of the present invention, a method for imaging a series of medical images by tomosynthesis with blur reduction with an imaging system comprising a radiation source configured to emit a total radiation dose, a detector, and a control unit configured to control the source is provided. The method comprises positioning, with the control unit, the source in at least two positions relative to the detector. The method also comprises emitting an individual radiation dose at least partially detected by the detector in each of the at least two positions. The method further comprises distributing, with the control unit, the total radiation dose among the individual radiation doses such that the strongest individual radiation doses are emitted by the source in positions corresponding to the beginning of an imaging session of the series of medical images.

According to another embodiment of the present invention, an imaging system for imaging medical images by tomosynthesis is provided. The imagining system comprises a radiation source configured to emit a total radiation dose; a detector; and a control unit. The control unit is configured to position the source in at least two positions relative to the detector, wherein the source emits an individual radiation dose at least partially detected by the detector in each of the at least two positions. The control unit is also configured to distribute the total radiation dose among the individual radiation doses such that the strongest individual radiation doses are emitted by the source in positions corresponding to the beginning of an imaging session of the series of medical images.

### BRIEF DESCRIPTION OF THE SEVERAL VIEWS OF THE DRAWINGS

Other aims, characteristics and advantages will emerge from the following detailed description in reference to the drawings given by way of illustration and non-limiting, in which:

FIG. 1 illustrates an example of a medical imaging system used for executing the method for imaging a series of medical images by tomosynthesis with blur reduction according an embodiment of the present invention;

FIG. 2 is a diagram showing the steps of an example of the method for imaging a series of medical images by tomosynthesis with blur reduction according an embodiment of the present invention;

FIG. 3 illustrates an example of distribution of a total radiation dose between different positions of the source of the imaging system of FIG. 1 according an embodiment of the present invention; and

FIG. 4 is a graph showing the likelihood of movement of the patient as a function of time according an embodiment of the present invention.

### DETAILED DESCRIPTION OF THE INVENTION

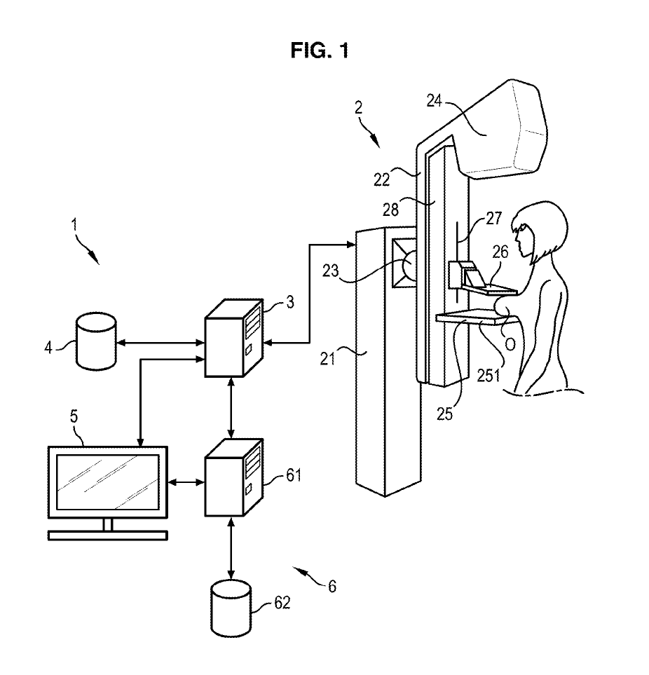

FIG. 1 schematically illustrates a medical imaging system 1 for the acquisition of images enabling three dimensions (3D) reconstruction of a breast O from two dimensions (2D) images of the breast O. The medical imaging system 1 is shown coupled to a calculation unit 6 for generating images revealing suspect zones of the breast O.

The medical imaging system 1 can be a device for mammography for detecting and characterising radiological signs in the event of the screening, diagnosis and the treatment of breast cancer (tissular matrix).

The medical imaging system 1 comprises an acquisition unit 2 of 2D images.

The acquisition unit 2 comprises a vertical support 21 and a positioning arm 22 connected to a radiation source 24; for example X-ray, and optionally a harmless light source dedicated to lighting during positioning of the breast O to be X-rayed. The positioning arm 22 is rotatably mounted on the vertical support 21 for rotating about a rotation shaft 23. The vertical support 21 is fixed. Therefore, rotation of the positioning arm 22 allows the source 24 to be positioned in different positions such that the main direction of emission of the source 24 in one position is different to that of the source 24 in another position.

The acquisition unit 2 also comprises a holding arm 28 fitted with a worktop comprising a breast support 25 and a compression pad 26 parallel to the breast support 25 for compressing the breast O when it is positioned on the breast support 25, as illustrated in FIG. 1. The compression pad 26 is positioned above the breast support 25 and can be translated relative to the latter along a translation rail 27. The breast support 25 comprises a radiation detector 251 corresponding to the radiation used by the source 24. The breast support 25 and compression pad 26 help keep the breast O immobile during acquisition of medical images.

The breast support 25 and the compression pad 26 can be planar. They can be positioned parallel to the floor or not, for example at 45° relative to the floor. The holding arm 28 can be mounted in rotation on the vertical support 21, advantageously about the same axis of rotation 23 as the positioning arm 22.

The positioning 22 and holding 28 arms are detached, allowing rotation of one relative to the other, and advantageously about the rotation shaft 23. They are positioned one relative to the other such that a large part of radiation emitted by the source 24 is received by the detector 251.

The detector 251 can be a semi-conductor image sensor comprising, for example, caesium iodide phosphorous (scintillater) on a matrix of transistors/photodiodes made of amorphous silicon. Other adequate detectors are: a CCD sensor or a direct digital detector which converts directly X-rays into digital signals. The detector illustrated in FIG. 1 is planar and defines a planar detection surface of a planar image. Other shapes are suitable, such as for example the digital X-ray detector with curved forms forming a curved image surface.

The medical imaging system 1 also comprises a control unit 3 connected to the acquisition unit 2 either by wired connection or via network. The control unit 3 sends electric controls signals to the acquisition unit 2 to set several parameters such as the radiation dose to be emitted, the angular positioning of the positioning arm 22, the angular positioning of the holding arm, the compression force the compression pad 26 has to apply to the breast O.

The control unit 3 can comprise a reading device (not shown), for example a disc reader, a CD-ROM, DVD-ROM reader, or connection ports for reading the instructions of the process for treating an instruction medium (not shown), such as a diskette, a CD-ROM, DVD-ROM, or USB stick or more generally by any removable memory medium or even via a network connection.

In another embodiment of the present invention, the control unit 3 can comprise a wired or wireless network connection device (not shown). In another embodiment, the control unit 3 executes the instructions of the treatment process stored in micro-software.

The medical imaging system 1 further comprises a memory unit 4 connected to the control unit 3 for recording the acquired parameters and images. It is possible to ensure that the database 4 is located inside the control unit 3 or outside it.

The memory unit 4 can be formed by a hard drive or SSD, or any other removable and rewritable storage means (USB sticks, memory cards, etc.).

The memory unit 4 can be ROM/RAM memory of the control unit 3, a USB sticks, a memory card, central server memory.

The medical imaging system 1 comprises a display 5 connected to the control unit 3 for displaying the acquired images and/or information on the parameters that the control unit 3 has to transmit to the acquisition unit 2.

The display 5 can be integrated into the acquisition unit 2 or the control unit 3 or even a calculation unit 6 described hereafter, or can be separated therefrom such as for example in the case of a review station used by the radiologist to conduct diagnosis from digital medical images.

The display 5 is for example a computer screen, a monitor, a flat screen, plasma screen or any type of commercially available display device.

The display 5 allows a practitioner to control reconstruction and/or display of the 2D images acquired.

The medical imaging system 1 is coupled to a calculation unit 6 comprising a 3D computer 61 which receives the acquired images and which are stored in the unit memory 4 of the medical imaging system 1, from which it constructs a 3D image of the breast O by digital tomosynthesis. An example of the process for breast digital tomosynthesis is described in greater detail in document FR 2 872 659.

The calculation unit 6 is for example a computer or computers, a processor or processors, a microcontroller or microcontrollers, a microcomputer or microcomputers, a programmable automaton or automatons, a specific application integrated circuit or circuits, other programmable circuits, or other devices including a computer such as a workstation.

The calculation unit 6 also comprises a memory unit 62 for storing data generated by the 3D computer 61.

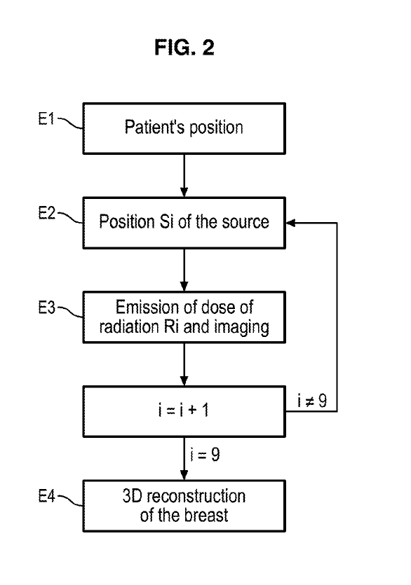

In reference to FIG. 2, it is described hereinbelow a method for imaging a series of medical images by tomosynthesis by means of a medical imaging system 1, for example the one described hereinabove. This process enables blur reduction resulting from movement of the patient during acquisition of medical images.

Prior to the process, the patient is positioned E1 relative to the acquisition unit 2 of the medical imaging system 1. In particular, the breast O to be X-rayed is placed on the breast support 25 and compressed by the compression pad 26.

When the patient is properly positioned and the breast O is held in place between the breast support 25 and the compression pad 26, a set of medical images is acquired with a radiation source 24 of the medical imaging system 1. For this, the source 24 is shifted E2 by the positioning arm 22 into different positions S₁-S₉ distributed around a circle C the centre Ω of which is contained in the detector 25.

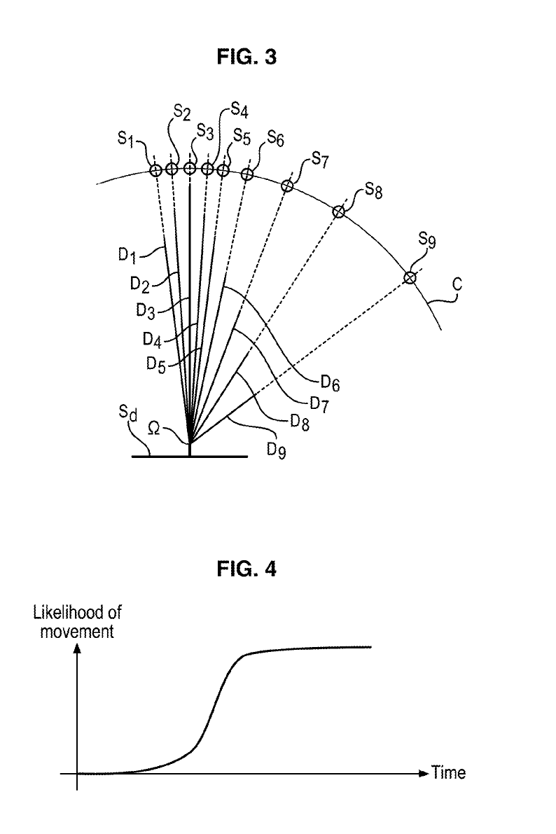

The control unit 3 distributes the total radiation dose Rₜₒₜ among different individual doses Rᵢ (Rₜₒₜ=Σᵢ Rᵢ) according to the positions S₁-S₉ of the source 24 and controls at the source 24 the emission E3 of the corresponding individual doses of radiation. The strongest individual doses of radiation Rᵢ are emitted by the source in the positions S₁-S₅ corresponding to the beginning of the imaging session of the series of medical images, as illustrated in FIG. 3.

In this FIG. 3, the detection surface S_{d} of the detector 251 is represented by a straight line, the main radiation directions D₁-D₉ of the source 24 also by straight lines. The main radiation directions D₁-D₉ correspond to positions S₁-S₉ of the source 24. The individual doses of radiation Rᵢ are symbolised by the length of the straight lines representing the main radiation directions D₁-D₉. The greater the length the stronger the individual radiation dose. The main directions D₁-D₉ are numbered according to the order of positioning of the source 24 during the imaging session of the series of medical images. Nine positions have been used here. The strongest individual doses of radiation correspond to the main directions D₁-D₅ or to the positions S₁-S₅.

The medical images acquired first will have a greater weight during reconstruction of the 3D image by the 3D computer 61 and their details will thus emerge on the 3D image. These medical images are taken while there is a low likelihood that the patient will move, as shown in FIG. 4, which represents the time in abscissa and the likelihood of movement of the patient in ordinates.

In fact, at the beginning of the imaging session, the patient concentrates and manages to contain her movements. But over time, her concentration can relax and movement is difficult to avoid. The medical images taken first thus have less risk of blurring due to movement of the patient than medical images taken later on.

The positions corresponding to the beginning of the imaging session of the series of medical images can be positions in which the source 24 emits radiation in a main direction which forms an angle close to 90° with the planar surface of the detector 251. Advantageously, the angle is between 80° and 100°, that is, with a straight line perpendicular to the surface of the detector 251 the main radiation direction forms an angle of between −10° and +10°. When the detection surface of the detector 251 is not planar, the angles are given relative to a medium plane of the detection surface of the detector 251. Tomosynthesis radiography is thus asymmetrical (see FIG. 3) and its advantage is obtaining medical images which are exposed more intensely and for which resolution is the finest and accordingly giving them more weight during reconstruction of the 3D image. Also, the medical image with the strongest individual radiation dose can be advantageously that taken with a main radiation direction of the source 24 perpendicular to the detection surface of the detector 251. Therefore, this image strongly resembles a conventional radiographic 2D image.

This also helps acquire medical images slightly before the main radiation direction of the source 24 is perpendicular to the surface of the detector 251.

In all cases, the sum of the strongest individual doses of radiation can be selected so as to be greater than 50% of the total radiation dose. By way of advantage, the sum of the strongest individual doses can be twice as great as the sum of the other individual doses.

The total radiation dose Rₜₒₜ can be distributed between the individual doses of radiation Rᵢ such that the individual doses of radiation Rᵢ decrease from one successive position Sᵢ to another Sᵢ₊₁ as a function of time or space.

The total radiation dose Rₜₒₜ can again be distributed between the individual doses R, such that the individual doses of radiation Rᵢ increase then decrease from one successive position Sᵢ to another Sᵢ₊₁, as illustrated in FIG. 3.

For example, nine positions of the source are defined as per the Table 1 below:

TABLE 1

| rank | angle | radiation |
|---|---|---|
| 1 | −6° | 10% |
| 2 | −3° | 15% |
| 3 | 0° | 25% |
| 4 | 3° | 15% |
| 5 | 6° | 10% |
| 6 | 9° | 8% |
| 7 | 12° | 7% |
| 8 | 15° | 5% |
| 9 | 18° | 5% |

“Rank” indicates the order of acquisition of the medical image, “angle” indicates the angle of the main direction of radiation relative to the normal at the surface of the detector and “radiation” indicates the percentage of the overall attributed radiation dose.

Finally, a 3D image on the breast O is reconstructed E4 by the 3D computer 61.

The process described hereinabove can be run by a computer program executed or running on a computer and which comprises adapted machine instructions.

The description was made in reference to mammography by X-ray. The tissular matrix is thus the breast. This choice barely reflects any limitation of the invention to application solely to mammography. The person skilled in the art will know how to adapt the teaching hereinabove described to any type of technique of acquisition of medical images allowing such.

What is claimed is:

1. A method for imaging a series of medical images by tomosynthesis with blur reduction with an imaging system comprising a radiation source configured to emit a total radiation dose, a detector, and a control unit configured to control the source, the method comprising:
positioning, with the control unit, the source in at least two positions relative to the detector;
emitting an individual radiation dose at least partially detected by the detector in each of the at least two positions; and
distributing, with the control unit, the total radiation dose among the individual radiation doses such that the strongest individual radiation doses are emitted by the source in positions corresponding to the beginning of an imaging session of the series of medical images.

2. The method as claimed in claim 1, wherein, in the positions corresponding to the beginning of the imaging session of the series of medical images, positioning, with the control unit, the source in at least two positions relative to the detector comprises positioning the source such that a main direction of emission of the source forms an angle of −10° to +10° with a straight line perpendicular to a detection surface of the detector.

3. The method as claimed in claim 1, wherein distributing, with the control unit, the total radiation dose comprises distributing the total radiation dose such that the sum of the strongest individual radiation doses is at least twice as strong as the sum of the other individual radiation doses.

4. The method as claimed in claim 1, wherein distributing, with the control unit, the total radiation dose comprises distributing the total radiation dose such that the individual radiation doses decrease from one successive position to another according to time.

5. The method as claimed in claim 1, wherein distributing, with the control unit, the total radiation dose comprises distributing the total radiation dose such that the individual radiation doses decrease from one successive position to another according to space.

6. The method as claimed in claim 1, wherein distributing, with the control unit, the total radiation dose comprises distributing the total radiation dose such that the individual radiation doses increase then decrease from one successive position to another.

7. An imaging system for imaging medical images by tomosynthesis, comprising:
a radiation source configured to emit a total radiation dose;
a detector; and
a control unit configured to:
position the source in at least two positions relative to the detector, wherein the source emits an individual radiation dose at least partially detected by the detector in each of the at least two positions; and
distribute the total radiation dose among the individual radiation doses such that the strongest individual radiation doses are emitted by the source in positions corresponding to the beginning of an imaging session of the series of medical images.

8. The imaging system as claimed in claim 7, wherein, in the positions corresponding to the beginning of the imaging session of the series of medical images, the control unit is configured to position the source such that a main direction of emission of the source forms an angle of −10° to +10° with a straight line perpendicular to a detection surface of the detector.

9. The imaging system as claimed in claim 7, wherein the control unit is further configured to distribute the total radiation dose such that the sum of the strongest individual radiation doses is at least twice as strong as the sum of the other individual radiation doses.

10. The imaging system as claimed in claim 7, wherein the control unit is further configured to distribute the total radiation dose such that the individual radiation doses decrease from one successive position to another according to time.

11. The imaging system as claimed in claim 7, wherein the control unit is further configured to distribute the total radiation dose such that the individual radiation doses decrease from one successive position to another according to space.

12. The imaging system as claimed in claim 7, wherein the control unit is further configured to distribute the total radiation dose such that the individual radiation doses increase then decrease from one successive position to another.
